(19) United States
(12) Patent Application Publication
(10) Pub. No.: US 2024/0035053 A1
(43) Pub. Date: Feb. 1, 2024

(54) Microbial Heavy Lanthanide Acquisition and Storage with Enhanced PQQ Production

(71) Applicant: The Regents of the University of California, Oakland, CA (US)

(72) Inventors: N. Cecilia Martinez-Gomez, Berkeley, CA (US); Nathan Michael Good, Berkeley, CA (US)

(73) Assignee: The Regents of the University of California, Oakland, CA (US)

(21) Appl. No.: 18/482,907

(22) Filed: Oct. 8, 2023

Related U.S. Application Data
(63) Continuation of application No. PCT/US2022/028235, filed on May 7, 2022.
(60) Provisional application No. 63/191,984, filed on May 22, 2021.

(51) Int. Cl. C12P 17/18; C12N 1/20; C12N 15/52; C12N 9/04; C12N 9/12

(52) U.S. Cl. CPC C12P 17/182; C12N 1/205; C12N 15/52; C12N 9/0006; C12N 9/12; C12N 2500/35; C12Y 207/13003

(57) ABSTRACT

Lanthanides are recycled with microbes comprising a lanthanide-dependent alcohol dehydrogenase and an evo-HLn Methylorubrum extorquens AM1 hybrid sensor histidine kinase/response regulator comprising a Leu151His substitution relative to the wild-type.

### Introduction

Gadolinium is a key component of magnetic resonance imaging (MRI) contrast agents that are critical tools for enhanced detection and diagnosis of tissue and vascular abnormalities. Untargeted post-injection deposition of gadolinium in vivo, and association with diseases like nephrogenic systemic fibrosis (NSF), has alerted regulatory agencies to re-evaluate their wide-spread use and generated calls for new, safer gadolinium-based contrast agents (GBCAs). Increasing anthropogenic gadolinium in surface water has also raised concerns of potential ecotoxicity and bioaccumulation in plants and animals. Methylotrophic bacteria can acquire, transport, store and use light lanthanides as part of a cofactor complex with pyrroloquinoline quinone (PQQ), an essential component of XoxF-type methanol dehydrogenases (MDHs). MDH catalyzes the oxidation of methanol to formaldehyde, a critical reaction for methylotrophic growth with methane and methanol.

### SUMMARY OF THE INVENTION

The invention provides microbial-based methods, compositions and systems for gadolinium recycling, using inexpensive growth substrates like methanol, with the option to produce the vitamin supplement pyrroloquinoline quinone (PQQ) as a value-added product. Practical applications of the invention include removal of gadolinium from medical waste and waste water; isolation of pure gadolinium (acquired from medical waste/waste water) to be reused for production of new MRI contrast agents; production of the vitamin PQQ. The invention also provides engineered bacteria for heavy lanthanide acquisition and storage.

In an aspect the invention provides a method of removing a lanthanide from a medium, comprising growing a microbe in the medium under conditions wherein the growing microbe acquires the lanthanide from the medium, the microbe comprising a lanthanide-dependent alcohol dehydrogenase and an evo-HLn Methylorubrum extorquens AM1 hybrid sensor histidine kinase/response regulator comprising a Leu151His substitution relative to the wild-type, GenBank: ACS39642.1; Ref Seq Accession WP_012752627.1.

In embodiments:

the medium comprises a growth substrate, such as methanol, ethanol or glycerol;

the lanthanide is a heavy lanthanide, selected from gadolinium and europium (atomic numbers 64 and 63, respectively);

the microbe is a Methylobacteriaceae species, including Methylobacterium species, such as Methylobacterium adhaesivum, Methylobacterium aminovorans, Methylobacterium aquaticum, Methylobacterium chloromethanicum, M. dichloromethanicum, Methylobacterium extorquens, Methylobacterium fujisawaense, Methylobacterium hispanicum, Methylobacterium isbiliense, Methylobacterium lusitanum, Methylobacterium mesophilicum, Methylobacterium nodulans, Methylobacterium organophilum, Methylobacterium podarium, Methylobacterium populi, Methylobacterium radiotolerans, Methylobacterium rhodesianum, Methylobacterium rhodinum, Methylobacterium suomiense, Methylobacterium thiocyanatum, Methylobacterium variabile, Methylobacterium zatmanii; and Methylorubrum species, such as Methylorubrum aminovorans, Methylorubrum extorquens, Methylorubrum podarium, Methylorubrum populi, Methylorubrum pseudosasae, Methylorubrum rhodesianum, Methylorubrum rhodinum, Methylorubrum salsuginis, Methylorubrum suomiense, Methylorubrum thiocyanatum and Methylorubrum zatmanii;

the microbe is a Methylorubrum extorquens;

the method further comprises, after acquisition of an amount of the lanthanide, isolating the microbe from the medium;

the method further comprises isolating the lanthanide from the microbe;

the microbe is grown under conditions wherein the microbe produces pyrroloquinoline quinone (PQQ);

the microbe is grown under conditions wherein the microbe produces pyrroloquinoline quinone (PQQ), and the method further comprises isolating the PPQ from the microbe;

the regulator is recombinant and/or transgenic to the microbe;

the microbe is selected from an engineered Methylobacteriaceae species (supra) comprising a transgenic and/or recombinant regulator;

the regulator is encoded by a regulator gene comprising 452T>A mutation that results in the Leu151His substitution; and/or

the microbe comprises a genome comprising one or both of SNPs: 69T>C and 114T>C (GenBank: ACS39451.1; GenBank: ACS40444.1).

In an aspect, the invention provides an engineered microbe for removing a lanthanide from a medium, the microbe comprising a lanthanide-dependent alcohol dehydrogenase and a transgenic and/or recombinant Methylorubrum extorquens AM1 hybrid sensor histidine kinase/response regulator comprising a Leu151His substitution relative to the wild-type.

In embodiments:

the microbe is selected from an engineered Methylobacteriaceae species (supra);

the microbe is a Methylorubrum extorquens; and/or

the microbe comprises a genome comprising one or both of SNPs: 69T>C and 114T>C (GenBank: ACS39451.1; GenBank: ACS40444.1).

In an aspect the invention provides use of a disclosed microbe comprising a lanthanide-dependent alcohol dehydrogenase and an evo-HLn Methylorubrum extorquens AM1 hybrid sensor histidine kinase/response regulator comprising a Leu151His substitution relative to the wild-type, GenBank: ACS39642.1; Ref Seq Accession WP_012752627.1, for the acquisition, storage and use of heavy lanthanides.

The invention encompasses all combinations of the particular embodiments recited herein, as if each combination had been laboriously recited.

Description of Particular Embodiments of the Invention

Unless contraindicated or noted otherwise, in these descriptions and throughout this specification, the terms “a” and “an” mean one or more, the term “or” means and/or and polypeptide sequences are understood to encompass opposite strands as well as alternative backbones described herein. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

Physiological Characterization of a Methylorubrum extorquens AM1 Genetic Variant Isolated From Methanol Growth With the Heavy Lanthanide Gadolinium

Abstract

Here we report robust gadolinium-dependent methanol growth of a genetic variant of M. extorquens AM1, named evo-HLn, for “evolved for heavy lanthanides”. Genetic adaptation of evo-HLn resulted in the capability to grow on methanol using the heavy lanthanide gadolinium or europium, correlating with increased xox1 promoter and XoxF MDH activities, heavy lanthanide transport and storage, and increased biosynthesis of pyrroloquinoline quinone (PQQ). Evo-HLn was able to grow on methanol using the GBCA Gd-DTPA as the sole gadolinium source, showing the utility of this strain for gadolinium recovery from medical waste and/or wastewater and for generating new GBCAs.

Introduction

Gadolinium (Gd³⁺; atomic number 64) is a versatile element that is widely used in various modern industries (1) but is perhaps best-known for its use as a contrast agent for MRI. Its seven unpaired electrons give Gd³⁺ unparalleled paramagnetic properties, making it the most effective agent for clinical application (2). Gd³⁺ alone is highly toxic to humans (3) and is therefore injected as a nine-coordinate ion chelated by an octadentate polyaminocarboxylate ligand with a water coligand (4). The stability of GBCAs makes them highly effective for intravenous delivery, and as a result they are used in an estimated 30 million MRI exams annually (5), with around half a billion doses administered thus far (6). GBCAs are excreted in urine post-injection, however, they are not innocuous. Over the past two decades, the development of NSF has been observed in GBCA injection patients with impaired renal function, resulting in joint pain, immobility and even death, (7-10). The last five years have generated rising alarm over the use of GBCAs with long-term retention found in the central nervous system, skin and bones of patients with normal kidney function (11-14). Anaphylactic shock and kidney failure have also been reported as possible outcomes of Gd³⁺ accumulation in tissues (15, 16). Unmetabolized, excreted GBCAs are also cause for concern as rising anthropogenic Gd³⁺ in surface water correlates with steadily increasing annual MRI exams worldwide (1). Due to the toxicity and rising concentrations of this microcontaminant, the potential health impacts on aquatic life and bioaccumulation in the food-chain deserve more attention, as do wastewater treatment strategies that are sufficient to remove Gd³⁺.

Gadolinium is a member of the lanthanide series of elements, a group that has recently been added as life metals. A broader understanding of the functions of lanthanides (Ln³⁺) in biology is slowly unfurling with discoveries of novel enzymes, metabolic pathways, and organisms dependent on these metals. Ln³⁺ are known to form a cofactor complex with the prosthetic group PQQ for some alcohol dehydrogenase enzymes (17). XoxF MDH from the methylotrophic bacterium Methylorubrum (formerly Methylobacterium) extorquens AM1 was the first reported Ln³⁺-dependent metallo-enzyme, and members of this diverse enzyme class are wide-spread in marine, fresh water, phyllosphere, and soil habitats (17-22). ExaF ethanol dehydrogenase was the first reported Ln³⁺-dependent enzyme with a preference for a multi-carbon substrate, and its discovery has led to the identification of related enzymes in non-methylotrophic bacteria (23, 24). Ln³⁺ are also known to influence metabolic pathways in methylotrophic and non-methylotrophic bacteria (25, 26). To date, all known Ln³⁺-dependent metallo-enzymes are from bacteria and coordinate the metal-PQQ complex for catalytic function. However, the physiological importance of PQQ stretches well-beyond the prokaryotes. Mammals, including humans (27), and plants (28) benefit from PQQ. Eukaryotes (29, 30) and archaea (31) produce PQQ-dependent enzymes, though there is still much to be discovered regarding their activities and function.

Evidence for the biological use of lanthanides (Ln³⁺) in bacteria was first reported as the stimulation of methanol growth and expression of PQQ-MDH activity in bacterial cultures grown with lanthanum (La³⁺; atomic number 57) or cerium (Ce³⁺, atomic number 58) (32, 33). At the time, Ln³⁺ were considered unavailable and unutilized for biological processes due to their insolubility in nature, and it was proposed that though Ln³⁺ are more potent Lewis acids than calcium (Ca²⁺), evolution likely passed them by in favor of the more bioavailable metal (34). PQQ-MDHs were typified by MxaFI, an α₂β₂ tetrameric enzyme that coordinates Ca²⁺ in the large subunit of each protomer (35, 36). MDH is a critical enzyme for methylotrophic bacteria, organisms that can oxidize reduced carbon compounds with no carbon-carbon bonds, such as methane and methanol, and has been the subject of genetic, biochemical and chemical studies for decades (37-42). Shortly after the discovery of Ln³⁺ dependence for XoxF MDH activity in M extorquens AM1 (18), the extremophile methanotroph Methylacidiphilum fumariolicum SolV was shown to rely on Ln³⁺ in its volcanic mudpot environment for survival (43). Several subsequent studies noted the role of Ln³⁺ in regulating MDH expression (44-46), describing the “lanthanide-switch” phenomenon in which the presence of light Ln³⁺ up-regulates expression of xox genes and concomitantly down-regulates expression of mxa genes. Global studies have suggested that Ln³⁺ may impact more than MDH and accessory gene expression, including alterations to downstream metabolism (25, 47, 48).

Growth studies with mesophilic methylotrophs and the Ln³⁺ series of metals have shown that only members of the “light” classification, ranging from La³⁺ to Nd³⁺ (atomic number 60), can support growth with XoxF MDH similar to Ca²⁺ with MxaFI MDH (49). In comparison, methanol growth with Sm³⁺ is much slower and growth has not been reported for Ln³⁺ of higher atomic numbers, with a couple of exceptions (20, 45, 50). M fumariolicum SolV is able to grow with the light/heavy lanthanide Eu³⁺ well enough to produce cultures for enzyme purification (51). This organism was also reported to show slow growth with Gd³⁺, but no studies have investigated this further (43). M fumariolicum SolV grows optimally in acidic conditions (pH 2-5) making Ln³⁺ soluble for uptake and utilization, and as such does not have a known dedicated transport system for these metals. In contrast, methylotrophs that grow at neutral pH have an ABC transport system and specific TonB-dependent receptor encoded in a “lanthanide-utilization and transport” gene cluster (22, 52). Of such organisms known to date, only a genetically manipulated mutant strain of Methylotenera mobilis JLW8 has been reported to show positive signs of growth with Gd³⁺ in the form of increased culture density (20). Thus, the heavy lanthanide Gd³⁺ is the highest atomic number species known to support methanol growth in methylotrophic bacteria. Activity of XoxF MDH decreases with increasing atomic radius for the light Ln³⁺(25, 51). While decreasing XoxF MDH activity correlates with reduced growth rates seen with Ln³⁺ of increasing atomic mass, it is still not known if this is due solely to decreased enzyme catalysis or if transport of the metal ions plays a role as well. Regardless of the factor(s) limiting growth, Gd³⁺ seems to be the pivotal Ln³⁺ marking the threshold of life with these metals.

Here we report the characterization of a M. extorquens AM1 genetic variant that is capable of robust growth on methanol with the heavy lanthanide Gd³⁺, a Ln³⁺ that does not support growth in the ancestral strain. The variant has a single base-pair genetic change in a putative hybrid histidine kinase/response regulator that has no previously-known link with methylotrophy or Ln³⁺ biochemistry. The variant exhibited increased xox1 promoter and MDH activities, a distinctive bright pink coloration corresponding to augmented PQQ production, and increased transport and storage of the metal. Further, genetic adaptation also allowed for faster growth with Sm³⁺ and growth with the light/heavy lanthanide Eu³⁺. Finally, we show that the variant could grow efficiently with the GBCA Gd-DTPA as the sole Ln³⁺ source. The invention provides methods and compositions for bioremediation and Ln³⁺ recycling, and genetically-encoded and peptide-based imaging agents.

### RESULTS

Isolation of an M. extorquens AM1 mutant strain capable of gadolinium-dependent methanol growth. The ΔmxaF mutant strain of M. extorquens AM1 has been reported to grow on methanol when provided an exogenous source of light lanthanides ranging from La³⁺ to Sm³⁺, but heavy lanthanides such as Eu³⁺ or Gd³⁺ were not included in the study (45). Gadolinium is the heaviest Ln³⁺ species that can be provided to produce a positive growth response (diminished growth compared to growth with light Ln³⁺) in a methylotroph to date (49). To better understand the limit of Ln³⁺-dependent methanol growth of M. extorquens AM1, we first tested the ability of ΔmxaF to grow on methanol with Gd³⁺ as the sole Ln³⁺ available. MP methanol minimal medium with Gd³⁺ was inoculated with ΔmxaF and culture density was measured over time. No detectable increase in culture density was observed after 14 days of incubation at 30° C. However, after another 7 days of incubation the culture density had increased ˜2.3 fold, reaching a final OD₆₀₀ of 0.35±0.03 (N=4). Gd³⁺-grown cells were transferred to fresh methanol minimal medium with Gd³⁺ and grown to maximum culture density. This process was repeated twice.

To verify that the cultures were not contaminated, 5 μL was plated onto solid minimal succinate medium with 50 μg/mL rifamycin and incubated at 30° C. Growth of pink colonies indicated the cultures were M. extorquens AM1, as the strain used is rifamycin-resistant (53). Using colony PCR, we determined that cells recovered from the Gd³⁺-grown cultures were negative for mxaF, as was the ancestral strain, and positive for fae encoding formaldehyde-activating enzyme, another genetic marker specific for M. extorquens AM1. Cells from these Gd^{3±}-grown cultures were washed four times with sterile minimal medium to remove possible residual extracellular Gd³⁺, resuspended in 1 mL sterile medium, and saved as freezer stocks with 5% DMSO at −80° C.

The long incubation time of the original cultures prior to growth with Gd³⁺ suggested either an extended period of metabolic acclimation or genomic adaptation. To discern between these two possibilities, we tested methanol growth after first passaging the strain three times on solid succinate medium and then inoculating into liquid succinate medium to generate pre-cultures. Cells from the liquid culture were harvested, washed four times with sterile minimal medium, and then inoculated into methanol medium with Gd³⁺. Growth was measured using a microplate spectrophotometer. The variant strain exhibited growth within ˜15 hours of inoculation, a specific growth rate of 0.03±0.001 h⁻¹, and a maximum culture density 0.69±0.04. The lack of the 3-week lag in growth, as we observed with the ancestral ΔmxaF inoculation, was indicative of genomic adaptation, rather than metabolic acclimation, being the underlying mechanism for growth with Gd³⁺.

Genomic DNA was isolated from the variant, sequenced, and analyzed for mutations relative to the wild type and ancestral ΔmxaF strains. Three single nucleotide polymorphisms (SNPs) were identified in the variant compared to ΔmxaF (Table S1). Only one of the three mutations was categorized as non-synonymous: a T to A nucleotide transition, resulting in a leucine to histidine amino acid substitution in a hybrid histidine kinase/response regulator. The mutation was confirmed by Sanger sequencing analysis, and the variant strain was named evo-HLn for “evolved for growth with heavy lanthanides”.

Increased PQQ biosynthesis. We observed that the cells of evo-HLn grown in methanol minimal medium with Gd³⁺ had a distinctive, bright pink coloration, and that extracts prepared from evo-HLn cells retained this increased pigmentation. When analyzed by UV-visible spectrophotometry, evo-HLn extracts displayed a unique peak at 361 nm. A peak around this wavelength is a signature of PQQ when bound to XoxF MDH or ExaF EtDH (25, 54). To confirm PQQ was the cause of the absorption anomaly, we spiked it into the evo-HLn extracts and observed an increase at the same wavelength. After normalizing for protein concentrations, the absorbance spectra indicated PQQ in evo-HLn extracts was 4-fold higher compared to wild type and 6-fold higher compared to ΔmxaF extracts.

Expanded range of lanthanide utilization for methanol growth. Robust methanol growth dependent on the heavy lanthanide Gd³⁺ was highly reproducible with evo-HLn, and we wondered if the acquired genetic adaptation(s) could impact the capacity for growth with other Ln³⁺. Compared to ancestral ΔmxaF, evo-HLn exhibited a statistically significant 22.9% slower growth rate on methanol with La³⁺ (One-way analysis of variance (ANOVA) p<0.001) (Table 1). This may be a result in a trade-off between the capacities to grow with light and heavy Ln 3+. We also tested for increased utilization of Sm³⁺, the highest atomic number Ln³⁺ shown to allow growth of ΔmxaF. Because it was reported that increasing the concentration of Sm³⁺ ten times to 20 μM resulted in more robust growth of ΔmxaF (faster growth rate, higher growth yield (45)), we tested the impact of this concentration on methanol growth of evo-HLn with Sm³⁺. Compared to ΔmxaF, evo-HLn grew at nearly double the rate reaching a maximum culture density similar to cultures grown with La³⁺ (Table 1). In addition, we tested for methanol growth of evo-HLn with the heavy lanthanides Eu³⁺ and Dy³⁺. Unlike ΔmxaF, evo-HLn was able to grow with Eu³⁺, though the growth rate and yield were reduced compared to both Sm³⁺ and Gd³⁺ (Table 1). evo-HLn did not show appreciable growth with Dy³⁺.

Increased xox1 promoter and MDH activities. Since XoxF MDH is closely-linked with Ln³⁺-dependent methanol growth, one plausible explanation for the expanded range of metals used by evo-HLn was increased XoxF MDH activity. Reporter-fusion assays previously showed that xox1 promoter activity was stimulated by light Ln³⁺ ranging from La³⁺ to neodymium (atomic number 60), with only a minor increase above background activity with Sm³⁺ (45). We measured xox1 promoter activity in evo-HLn with La³⁺ and observed a 7-fold increase compared to ΔmxaF and an 11-fold increase compared to wild type. Next, we measured xox1 promoter activity with Gd³⁺ from evo-HLn and observed a similar increase. Further, we did not detect xox1 promoter activity in wild type with Gd³⁺, showing that although the wild type grows with methanol in the presence of Gd³⁺, the regulatory switch from MxaFI MDH to XoxF MDH oxidation systems does not occur. This could be indicative of either the wild type being unable to transport Gd³⁺ or Gd³⁺ not functioning as a signal for the “lanthanide switch” in this strain. Regardless, it can be concluded that wild type grows on methanol using MxaFI MDH, the Ca²⁺/PQQ-dependent oxidation system, when Gd³⁺ is present in the medium.

Next, we measured MDH activity in cell-free extracts of ΔmxaF and evo-HLn prepared from cultures grown with methanol and either La³⁺ or Gd³⁺. When grown with La³⁺, MDH activity in evo-HLn extracts was ˜3-fold higher than in ΔmxaF extracts, verifying increased production of XoxF enzyme. Ln³⁺ species do not function equally well as part of the XoxF MDH cofactor complex, along with PQQ, and the enzyme active site is finely tuned for light Ln³⁺ (49, 51). Therefore, a reduction in XoxF MDH function could be expected with Gd³⁺ in the active site. MDH activity was detectable in extracts of evo-HLn grown with Gd³⁺ corresponding to 68% of the activity measured in extracts of ΔmxaF with La³⁺. evo-HLn grows well with Gd³⁺, and increased production of XoxF MDH is likely a major contributor to this metabolic capability. Increased xox1 promoter and MDH activities of evo-HLn are indicative of increases in Ln³⁺ transport and intracellular accumulation.

Enhanced Lanthanide accumulation in evo-HLn. Using inductively-coupled plasma optical emission spectroscopy (ICP-OES), we determined the Ln³⁺ metal content of cells grown with methanol and a single Ln³⁺ element species. We measured a significant (Student's t-test, p <0.05; n=3) 57% increase in Gd³⁺ from evo-HLn compared to La³⁺ from ΔmxaF. This increase is striking, and to our knowledge, the first report of enhanced Ln³⁺ uptake and intracellular storage in a methylotroph.

Efficient acquisition of Gd³⁺ from the GBCA Gd-DTPA. Finally, the capacity of the evo-HLn strain to acquire Gd³⁺ from the chelator diethylenetriamine pentaacetate (DTPA) was demonstrated. Despite the high stability of the Gd-DTPA complex (log Kₜₕₑᵣₘ 22, log K_{cond}) 17; (55, 56)), evo-HLn was able to grow readily with no reduction growth rate compared to growth with soluble GdCl₃ (Gd-DTPA, 0.04 h⁻¹±0.00; GdCl3, 0.03 h⁻¹±0.00; n=3). This result indicates that evo-HLn has a highly effective means of sequestering Gd³⁺ from DTPA, thus demonstrating a potential importance as a key player in Gd³⁺ recycling and pollution remediation.

Materials and Methods

Strains and culture conditions. M extorquens AM1 strains were routinely grown at 30° C. MP minimal medium (65) with 15 mM succinate, shaking at 200 rpm on an Innova 2300 platform shaker (Eppendorf, Hamburg, Germany). For growth studies, 50 mM methanol was used as the sole carbon and energy source. Lanthanides were added as chloride salts or gadopentetic acid (Gd-DTPA; Magnevist®□) to a working concentration of 2 or 20 μM as indicated. When necessary, 50 μg/mL kanamycin was added to the growth medium for plasmid maintenance. Strains and plasmids used in this study are listed in Table S1 of the supplementary material.

Strain construction. M. extorquens AM1 strains were transformed by electroporation (66). After 24 hours of outgrowth transformants were selected by plating on MP medium with 1.5% agar, 15 mM succinate and 50 μg/mL kanamycin. Transformants grew for 72 hours at 30° C. until individual colonies appeared.

Methanol growth analysis with light and heavy lanthanides. M extorquens AM1 strains were grown with succinate overnight, cells were pelleted by centrifugation at 1,000×g for 10 min at room temperature using a Sorvall Legend X1R centrifuge (Thermo Scientific, Waltham, MA, USA), and washed in 1 mL of sterile MP medium with methanol. For growth analysis in microplates, washed cells were resuspended in 200 μL of MP methanol medium and 10 μL were transferred to each microplate well with 0.64 μL MP methanol medium for inoculation. For growth studies with Gd-DTPA, 50 μL of inoculum was added to 3 mL MP methanol medium in sterile 14 mL polypropylene culture tubes (Fisher Scientific, Hampton, NH, USA). Cultures densities were monitored over time by measuring light scatter at 600 nm using either a Synergy HTX multi-mode plate reader (Biotek, Winooski, VT, USA) or an Ultraspec 10 density meter (Biochom, Holliston, MA, USA).

UV-visible spectrophotometry. To prepare cell-free extracts, 50 mL of methanol grown culture with Gd³⁺ or La³⁺ was harvested, upon reaching an OD₆₀₀ of ˜1.1-1.3, by centrifugation at 4,696×g for 10 minutes at 4° C. The supernatant was removed and cell pellets were resuspended in 1.5 mL of 25 mM Tris, pH 8.0 and lysed using an OS Cell Disrupter at 25,000 psi (Constant Systems Limited, Low March, Daventry, Northants, United Kingdom). Lysates were transferred to 1.5 mL eppendorf tubes and clarified of cell debris by centrifugation at 21,000×g for 10 minutes at 4° C. Cell-free extracts were transferred to new eppendorf tubes and kept on ice until needed. PQQ was prepared fresh to a working concentration of 5.3 mM in an opaque conical tube and kept on ice until needed. Absorbance spectra were measured from 250-600 nm with a Synergy HTX multi-mode plate reader. A blank buffer spectrum was subtracted as background. Protein concentrations were determined by absorbance at 280 nm and the bicinchoninic acid assay (ThermoFisher Scientific, Waltham, MA, USA).

Genomic DNA extraction and sequencing. The ΔmxaF and ΔmxaF_Gd mutant strains were grown in shake flasks with 50 mL MP with succinate to early exponential growth phase. Cultures were transferred to 50 mL conical tubes and cells were harvested by centrifugation using a Sorvall Legend XR1 centrifuge at 4,696×g, 4° C. for 10 mM The supernatant was removed and the cell pellets were resuspended in 30 mL of TE buffer (10 mM Tris, 1 mM EDTA, pH 8.0) before transferring to a new conical tube. After adding 80 mg of lysozyme, samples were mixed by vortexing and then incubated at 37° C. Next, 1.6 mL of 10% sodium dodecyl sulfate and 1 mg proteinase K were added to the samples, vortexing after each addition, and then samples were incubated at 56° C. for 16 hours. After, 4 mL of 5 M NaCl was added and samples were mixed by vortexing. Then, 4 mL of 2.2 mM hexadecyltrimethylammonium bromide with 5.6 mM NaCl pre-heated to 65° C. was added and samples were vortexed. Samples were split evenly between two 50 mL conical tubes and incubated for 10 minutes at 65° C. Next, 20 mL of phenol:chloroform:isoamyl alcohol (25:24:1) was added, samples were mixed by vortexing, and then spun at 4,696×g for 10 minutes at room temperature. The aqueous phase was then transferred to a new conical tube and 20 mL chloroform:isoamyl alcohol (24:1) was added. Samples were mixed and spun at 4,696×g for 10 minutes at room temperature, after which the aqueous phase was transferred to a clean conical tube. Isopropanol chilled at −20° C. was then added to each sample at a ratio of 0.6:1.0, samples were mixed and then incubated at −20 C. for 16 hours. Samples were then spun at 4,696×g for 45 minutes at 4° C. and the supernatant was removed. Pellets were washed with ice cold 70% ethanol and then spun at 4,696×g for 5 minutes at 4° C. The supernatant was discarded and the pellets were dried at room temperature. DNA samples were then treated for RNA contamination by resuspending each pellet in 170 μL of DNase-free water, adding RNase I and incubating at 37° C. for 1 hour. After 1 hour, 5 μL of each RNase I-treated and untreated sample were analyzed by gel-electrophoresis for trace RNA. After verifying that RNA was degraded, the RNase I was inactivated by heating the samples at 70° C. for 15 minutes. Samples were then cooled on ice and 20 ηL, 3M sodium acetate and 550 μL 100% ethanol were added. After mixing, samples were incubated overnight at −20° C. DNA was pelleted by spinning the samples for 20 minutes at 21,000×g and 4° C., after which the supernatant was carefully poured off. DNA pellets were washed with ice-cold 70% ethanol and then spun at 21,000×g and 4° C. for 5 minutes. Ethanol was removed by carefully pipetting and the DNA pellets were then air dried at room temperature. Finally, DNA samples were resuspended in 100 μL of DNase-free water. Samples were submitted to Genewiz (South Plainfield, NJ, USA) for whole genome sequencing using the Illumina HiSeq platform with 2×150 bp read length. Variant calling and analysis was performed by Genewiz.

Transcriptional reporter fusion assays. Strains carrying VENUS yfp fusion constructs were grown on methanol in 48-well microplate format. Upon reaching a culture density of OD₆₀₀ ˜0.35, 200 μL of culture were transferred to an optical bottom black 96-well plate. Fluorescence was measured at an excitation wavelength of 485 nm and an emission wavelength of 520 nm. Relative fluorescence units (RFU) were calculated as raw fluorescence divided by OD₆₀₀.

Methanol dehydrogenase activity assays. Cell extracts were prepared as described above, but with an additional wash step in 20 mL of 100 mM Tris-HCl, pH 9.0 before lysing. Protein concentrations of cell-free extracts were determined by BCA assay. Methanol dehydrogenase activity was measured by monitoring the phenazine methosulfate (PMS)-mediated reduction of 2,6-dichlorophenol indophenol (DCPIP; ϵ₆₆₀ₙₘ=21 mM⁻¹ cm⁻¹ (25, 54, 67)) as described (45, 67-69). To reduce background activity, all assay reagents were dissolved in water; PES and DCPIP solutions were prepared in opaque tubes and kept on ice; and cell-free extracts were pre-incubated for 2 minutes at 30° C. as recommended (70).

Intracellular Ln³⁺ quantification. After whole-cell MRI analysis, cell pellets were dehydrated at 65° C. for 72 hours. Dried pellets were weighed before deconstruction in Aqua regia diluted in 2% nitric acid, and sonicated for 0.5 h before passing through 0.5 μm Whatman syringe filters. Metal contents were determined by ICP-OES using a Varian 710-ES ICP-OES (Santa Clara, CA, USA) with standard solutions purchased from Sigma-Aldrich.

TABLE 1
Growth rates and yields of strains grown in minimal medium with
methanol Ln³⁺. Culture density was monitored for up to 96 hours.

| strain | Ln³⁺ source^{ϵ} | growth rate^{ϑ} | growth yield^{ϑ} |
|---|---|---|---|
| wild type | none | 0.15 ± 0.01 | 0.72 ± 0.13 |
| wild type | LaCl₃ | 0.15 ± 0.01 | 0.78 ± 0.14 |
| wild type | GdCl₃ | 0.14 ± 0.00 | 0.77 ± 0.14 |
| ΔmxaF | none | n.d. | — |
| ΔmxaF | LaCl₃ | 0.14 ± 0.01 | 0.90 ± 0.04 |
| ΔmxaF | SmCl₃^{#} | 0.03 ± 0.01^{§} | 0.20 ± 0.01^{§} |
| ΔmxaF | EuCl₃^{#} | n.d.^{§} | —^{§} |
| ΔmxaF | GdCl₃ | n.d. | — |
| ΔmxaF | DyCl₃^{#} | n.d.^{§} | —^{§} |
| evo-HLn | none | n.d. | — |
| evo-HLn | LaCl₃ | 0.11 ± 0.01 | 0.93 ± 0.13 |
| evo-HLn | SmCl₃^{#} | 0.06 ± 0.00 | 0.89 ± 0.10^{§} |
| evo-HLn | EuCl₃^{#} | 0.05 ± 0.00^{§} | 0.25 ± 0.01^{§} |
| evo-HLn | GdCl₃ | 0.03 ± 0.00 | 0.69 ± 0.04 |
| evo-HLn | DyCl₃^{#} | n.d.^{§} | —^{§} |

^{ϵ}2 μM LnCl₃ provided as the sole source of Ln³⁺ except where indicated
^{#}20 μM LnCl₃ provided as the sole source of Ln³⁺
^{ϑ}Values represent the averages of 10 biological replicates from 3 independent experiments except where indicated. Error bars are standard errors of the mean (SEM). n.d. is not determined. - is no growth.
^{§}Values are the mean of 5 biological replicates from two independent experiments.

TABLE S1
Bacterial strains and plasmids

| | | |
|---|---|---|
| strain or | | |
| plasmid strains | description | reference |

Methylorubrum extorquens

| | | |
|---|---|---|
| AM1 | wild type; rifamycin-resistant derivative | (1) |
| ΔmxaF | deletion mutant | (2) |
| evo-HLn | ΔmxaF deletion mutant variant adapted for | this study |
| | methanol growth with heavy lanthanides | |

plasmids

| | | |
|---|---|---|
| pNG326 | P_{L}/O4/A1 expression vector with evo-HLn | this study |
| | variant META1_1800 allele, Km^{r} | |
| pAP05 | promoterless yfp fusion vector, Tc^{r} | (3) |
| pES503 | pAP05 with xox1 promoter region, Tc^{r} | (3) |

TABLE S2
Mutations detected by genome resequencing of ΔmxaF and evo-HLn
Wild-type M. extorquens AM1 was used as the reference strain for mapping. Green, mutations unique to ΔmxaF; yellow, mutations unique toevo-HLn.

| | | | | | | | | | | Amino | |
|---|---|---|---|---|---|---|---|---|---|---|---|
| Chromo- | | | | | | | | | Coding | acid | |
| some | Region | Type | Ref | Allele | Count | Freq | Qual | locus_tag | change | change | Non |
| CP001510 | 482893\hat{ }482894 | In | — | C | 47 | 100 | 200 | META1p0458 | | | — |
| CP001510 | 1673173 | SNV | A | G | 110 | 100 | 200 | META1p1592 | 69T > C | | No |
| CP001510 | 1873778 | SNV | T | A | 175 | 100 | 200 | META1p1800 | 452T > A | Leu151His | Yes |
| CP001510 | 2329711 | Del | G | — | 125 | 98 | 160 | | | | — |
| CP001510 | 2777457 | SNV | T | G | 38 | 97 | 160 | META1p2648 | 408A > C | | No |
| CP001510 | 2803789 | SNV | C | T | 32 | 100 | 200 | META1p2676 | .63C > T | | No |
| CP001510 | 2803840 | SNV | T | C | 9 | 100 | 155 | META1p2676 | 114T > C | | No |
| CP001510 | 2891642 | SNV | G | C | 185 | 100 | 200 | META1p2763 | 879C > G | | No |
| CP001510 | 3037769 | Del | C | — | 190 | 95 | 200 | META1p2908 | 718delC | Arg241fs | Yes |
| CP001510 | 3159071 | Del | G | — | 94 | 97 | 160 | | | | — |
| CP001510 | 4001527 . . . 4001531 | Del | CGTGC | — | 122 | 85 | 200 | META1p3891, | 262_266delGCACG | Ala88fs | Yes |
| | | | | | | | | META1p3892 | | | |
| CP001510 | 4322600 | SNV | G | A | 4 | 67 | 26 | META1p4234 | 960C > T | | No |
| CP001511 | 580985\hat{ }580986 | In | — | C | 128 | 98 | 200 | META2p0619 | 468_469insG | Arg157fs | Yes |
| CP001511 | 770863 | Del | G | — | 52 | 95 | 160 | META2p0816 | 890delG | Ala298fs | Yes |

### REFERENCES

1. Ebrahimi P, Barbieri M. 2019. Gadolinium as an Emerging Microcontaminant in Water Resources: Threats and Opportunities. Geosci J 9:93.

2. Srivastava A K, et al. 2015. Advances in using MRI probes and sensors for in vivo cell tracking as applied to regenerative medicine. Dis Model Mech 8:323-336.

3. Le Fur M, Caravan P. 2019. The biological fate of gadolinium-based MRI contrast agents: a call to action for bioinorganic chemists. Metallomics 11:240-254.

4. Wahsner J, Gale EM, Rodríguez-Rodríguez A, Caravan P. 2019. Chemistry of MRI Contrast Agents: Current Challenges and New Frontiers. Chem Rev 119:957-1057.

5. Lohrke, J. et al. 25 Years of Contrast-Enhanced MRI: Developments, Current Challenges and Future Perspectives. Adv Ther 33,1-28 (2016).—Google Search.

6. McDonald R J, et al. 2018. Gadolinium Retention: A Research Roadmap from the 2018 NIH/ACR/RSNA Workshop on Gadolinium Chelates. Radiology 289:517-534.

7. Grobner T. 2006. Gadolinium—a specific trigger for the development of nephrogenic fibrosing dermopathy and nephrogenic systemic fibrosis? Nephrol Dial Transplant 21:1104-1108.

8. Marckmann P, et al. 2006. Nephrogenic systemic fibrosis: suspected causative role of gadodiamide used for contrast-enhanced magnetic resonance imaging. J Am Soc Nephrol 17:2359-2362.

9. Boyd AS, Zic JA, Abraham JL. 2007. Gadolinium deposition in nephrogenic fibrosing dermopathy. J Am Acad Dermatol 56:27-30.

10. High WA, et al. 2007. Gadolinium is detectable within the tissue of patients with nephrogenic systemic fibrosis. J Am Acad Dermatol 56:21-26.

11. Kanda T, et al. 2014. High signal intensity in the dentate nucleus and globus pallidus on unenhanced Tl-weighted MR images: relationship with increasing cumulative dose of a gadolinium-based contrast material. Radiology 270:834-841.

12. Kanda T, et al. 2015. Gadolinium-based Contrast Agent Accumulates in the Brain Even in Subjects without Severe Renal Dysfunction: Evaluation of Autopsy Brain Specimens with Inductively Coupled Plasma Mass Spectroscopy. Radiology 276:228-232.

13. McDonald RJ, et al. 2015. Intracranial Gadolinium Deposition after Contrast-enhanced MR Imaging. Radiology 275:772-782.

14. Roberts DR, et al. 2016. High Levels of Gadolinium Deposition in the Skin of a Patient With Normal Renal Function. Invest Radiol 51:280-289.

15. Ergtin I, et al. 2006. The safety of gadolinium in patients with stage 3 and 4 renal failure. Nephrol Dial Transplant 21:697-700.

16. Hasdenteufel F, et al. 2008. Anaphylactic shock after first exposure to gadoterate meglumine two case reports documented by positive allergy assessment. J Allergy Clin Immunol 121:527-528.

17. Keltjens J T, Pol A, Reimann J, Op den Camp HJM. 2014. PQQ-dependent methanol dehydrogenases: rare-earth elements make a difference. Appl Microbiol Biotechnol 98:6163-6183.

18. Nakagawa T, et al. 2012. A catalytic role of XoxF1 as La3+-dependent methanol dehydrogenase in Methylobacterium extorquens strain AM1. PLoS One 7:e50480.

19. Taubert M, et al. 2015. XoxF encoding an alternative methanol dehydrogenase is widespread in coastal marine environments. Environ Microbiol 17:3937-3948.

20. Huang J, et al. 2018. Lanthanide-Dependent Methanol Dehydrogenases of XoxF4 and XoxF5 Clades Are Differentially Distributed Among Methylotrophic Bacteria and They Reveal Different Biochemical Properties. Front Microbiol 9:1366.

21. Chistoserdova L. 2016. Lanthanides: New life metals? World J Microbiol Biotechnol 32:138.

22. Ochsner A M, et al. 2019. Use of rare-earth elements in the phyllosphere colonizer Methylobacterium extorquens PAl. Mol Microbiol 111:1152-1166.

23. Wehrmann M, et al. 2017. Functional Role of Lanthanides in Enzymatic Activity and Transcriptional Regulation of Pyrroloquinoline Quinone-Dependent Alcohol Dehydrogenases in Pseudomonas putida KT2440. MBio 8:e00570-17.

24. Wegner C-E, et al. 2019. Lanthanide-Dependent Methylotrophs of the Family Beijerinckiaceae: Physiological and Genomic Insights. Appl Environ Microbiol 86:1-18.

25. Good N M, et al. 2019. Contrasting in vitro and in vivo methanol oxidation activities of lanthanide-dependent alcohol dehydrogenases XoxF1 and ExaF from Methylobacterium extorquens AM1. Sci Rep 9:4248.

26. Wehrmann M, et al. 2020. The Cellular Response to Lanthanum Is Substrate Specific and Reveals a Novel Route for Glycerol Metabolism in Pseudomonas putida KT2440. MBio 11.

27. Killgore J, et al. 1989. Nutritional importance of pyrroloquinoline quinone. Science 245:850-852.

28. Choi 0, et al. 2008. Pyrroloquinoline quinone is a plant growth promotion factor produced by Pseudomonas fluorescens B16. Plant Physiol 146:657-668.

29. Takeda K, et al. 2015. Characterization of a novel PQQ-dependent quinohemoprotein pyranose dehydrogenase from Coprinopsis cinerea classified into auxiliary activities family 12 in carbohydrate-active enzymes. PLoS One 10:e0115722.

30. Matsumura H, et al. 2014. Discovery of a eukaryotic pyrroloquinoline quinone-dependent oxidoreductase belonging to a new auxiliary activity family in the database of carbohydrate-active enzymes. PLoS One 9:e104851.

31. Sakuraba H, et al. 2010. Catalytic properties and crystal structure of quinoprotein aldose sugar dehydrogenase from hyperthermophilic archaeon Pyrobaculum aerophilum. Arch Biochem Biophys 502:81-88.

32. Hibi Y, et al. 2011. Molecular structure of La3+-induced methanol dehydrogenase-like protein in Methylobacterium radiotolerans. J Biosci Bioeng 111:547-549.

33. Fitriyanto NA, et al. 2011. Ce3+-induced exopolysaccharide production by Bradyrhizobium sp. MAFF211645. J Biosci Bioeng 111:146-152.

34. Lim S, Franklin SJ. 2004. Lanthanide-binding peptides and the enzymes that Might Have Been. Cell Mol Life Sci 61:2184-2188.

35. Richardson IW, Anthony C. 1992. Characterization of mutant forms of the quinoprotein methanol dehydrogenase lacking an essential calcium ion. Biochem J 287 (Pt 3):709-715.

36. Adachi O, et al.1990. Purification and Properties of Methanol Dehydrogenase and Aldehyde Dehydrogenase from Methylobacillus glycogenes. Agric Biol Chem 54:3123-3129.

37. Anthony C, Williams P 2003. The structure and mechanism of methanol dehydrogenase. Biochim Biophys Acta 1647:18-23.

38. Zhang X, Reddy SY, Bruice TC. 2007. Mechanism of methanol oxidation by quinoprotein methanol dehydrogenase. Proc Natl Acad Sci U S A 104:745-749.

39. Zheng Y-J, Xia Z-X, Chen Z-W, Scott Mathews F, Bruice T C. 2001. Catalytic mechanism of quinoprotein methanol dehydrogenase: A theoretical and x-ray crystallographic investigation. Proc Natl Acad Sci U S A 98:432-434.

40. Goodwin M G, Anthony C. 1996. Characterization of a novel methanol dehydrogenase containing a Ba2+ion at the active site. Biochem J 318 (Pt 2):673-679.

41. Williams P A, et al. 2005. The atomic resolution structure of methanol dehydrogenase from Methylobacterium extorquens. Acta Crystallogr D Biol Crystallogr 61:75-79.

42. Ghosh M, et al. 1995. The refined structure of the quinoprotein methanol dehydrogenase from Methylobacterium extorquens at 1.94 A. Structure 3:177-187.

43. Pol A, et al. 2014. Rare earth metals are essential for methanotrophic life in volcanic mudpots. Environ Microbiol 16:255-264.

44. Farhan Ul et al., 2015. Cerium regulates expression of alternative methanol dehydrogenases in Methylosinus trichosporium OB3b. Appl Environ Microbiol 81:7546-7552.

45. Vu HN, et al. 2016. Lanthanide-Dependent Regulation of Methanol Oxidation Systems in Methylobacterium extorquens AM1 and Their Contribution to Methanol Growth. J Bacteriol 198:1250-1259.

46. Chu F, Lidstrom M E. 2016. XoxF Acts as the Predominant Methanol Dehydrogenase in the Type I Methanotroph Methylomicrobium buryatense. J Bacteriol 198:1317-1325.

47. Gu W, et al. 2016. Uptake and effect of rare earth elements on gene expression in Methylosinus trichosporium OB3b. FEMS Microbiol Lett 363:1-20.

48. Masuda S, et al. 2018. Lanthanide-Dependent Regulation of Methylotrophy in Methylobacterium aquaticum Strain 22A. mSphere 3:e00462-17.

49. Daumann U. 2019. Essential and Ubiquitous: The Emergence of Lanthanide Metallobiochemistry. Angew Chem Int Ed Engl 58:12795-12802.

50. Wang L, et al. 2019. Lanthanide-dependent methanol dehydrogenase from the legume symbiotic nitrogen-fixing bacterium Bradyrhizobium diazoefficiens strain USDA110. Enzyme Microb Technol 130:109371.

51. Jahn B, et al. 2018. Similar but Not the Same: First Kinetic and Structural Analyses of a Methanol Dehydrogenase Containing a Europium Ion in the Active Site. Chembiochem https://doi.org/10.1002/cbic.201800130.

52. Roszczenko-Jasińska P, et al. 2020. Gene products and processes contributing to lanthanide homeostasis and methanol metabolism in Methylorubrum extorquens AM1. Sci Rep 10:12663.

53. Nunn D N, Lidstrom M E. 1986. Isolation and complementation analysis of 10 methanol oxidation mutant classes and identification of the methanol dehydrogenase structural gene of Methylobacterium sp. strain AM1. J Bacteriol 166:581-590.

54. Good N M, et al. 2016. Pyrroloquinoline Quinone Ethanol Dehydrogenase in Methylobacterium extorquens AM1 Extends Lanthanide-Dependent Metabolism to Multicarbon Substrates. J Bacteriol 198:3109-3118.

55. Tweedle M F, Hagan JJ, Kumar K, Mantha S, Chang C A. 1991. Reaction of gadolinium chelates with endogenously available ions. Magn Reson Imaging 9:409-415.

56. Wedeking P, Kumar K, Tweedle M F. 1992. Dissociation of gadolinium chelates in mice: relationship to chemical characteristics. Magn Reson Imaging 10:641-648.

57. Caravan P. 2006. Strategies for increasing the sensitivity of gadolinium based MRI contrast agents. Chem Soc Rev 35:512-523.

58. Dafni H, et al. 2002. Overexpression of vascular endothelial growth factor 165 drives peritumor interstitial convection and induces lymphatic drain: magnetic resonance imaging, confocal microscopy, and histological tracking of triple-labeled albumin. Cancer Res 62:6731-6739.

59. Gilad A A, et al. 2005. Functional and molecular mapping of uncoupling between vascular permeability and loss of vascular maturation in ovarian carcinoma xenografts: the role of stroma cells in tumor angiogenesis. Int J Cancer 117:202-211.

60. Xue S, et al. 2015. Protein MRI contrast agent with unprecedented metal selectivity and sensitivity for liver cancer imaging. Proc Natl Acad Sci U S A 112:6607-6612.

61. Sherry A D, Caravan P, Lenkinski R E. 2009. Primer on gadolinium chemistry. J Magn Reson Imaging 30:1240-1248.

62. Thomsen H S. 2017. Are the increasing amounts of gadolinium in surface and tap water dangerous? Acta radiol 58:259-263.

63. Cotruvo J A Jr, et al. 2018. Lanmodulin: A Highly Selective Lanthanide-Binding Protein from a Lanthanide-Utilizing Bacterium. J Am Chem Soc 140:15056-15061.

64. Lumpe H, Daumann L J. 2019. Studies of Redox Cofactor Pyrroloquinoline Quinone and Its Interaction with Lanthanides(III) and Calcium(II). Inorg Chem 58:8432-8441.

65. et al. 2013. Development of an optimized medium, strain and high-throughput culturing methods for Methylobacterium extorquens. PLoS One 8:e62957.

66. Toyama H, et al. ME. 2006. Construction of insertion and deletion mxa mutants of Methylobacterium extorquens AM1 by electroporation. FEMS Microbiol Lett 166:1-7.

67. Good N M, Fellner M, Demirer K, Hu J, Hausinger R P, Martinez-Gomez N C. 2020. Lanthanide-dependent alcohol dehydrogenases require an essential aspartate residue for metal coordination and enzymatic function. J Biol Chem 295:8272-8284.

68. Anthony C, Zatman L J. 1967. The microbial oxidation of methanol. The prosthetic group of the alcohol dehydrogenase of Pseudomonas sp. M27: a new oxidoreductase prosthetic group. Biochem J 104:960-969.

69. Ghosh R, Quayle J R. 1979. Phenazine ethosulfate as a preferred electron acceptor to phenazine methosulfate in dye-linked enzyme assays. Anal Biochem 99:112-117.

70. Jahn B, Jonasson NSW, et al. 2020. Understanding the chemistry of the artificial electron acceptors PES, PMS, DCPIP and Wurster's Blue in methanol dehydrogenase assays. J Biol Inorg Chem https://doi.org/10.1007/s00775-020-01752-9.

### SUPPL REFERENCES

1. Nunn D N, Lidstrom M E. 1986. Isolation and complementation analysis of 10 methanol oxidation mutant classes and identification of the methanol dehydrogenase structural gene of Methylobacterium sp. strain AM1. J Bacteriol 166:581-590.

2. Marx C J, O'Brien B N, Breezee J, Lidstrom M E. 2003. Novel methylotrophy genes of Methylobacterium extorquens AM1 identified by using transposon mutagenesis including a putative dihydromethanopterin reductase. J Bacteriol 185:669-673.

3. Skovran E, et al. 2011. XoxF is required for expression of methanol dehydrogenase in Methylobacterium extorquens AM1. J Bacteriol 193:6032-6038.

1. A method of removing a lanthanide from a medium, comprising growing a microbe in the medium under conditions wherein the growing microbe acquires the lanthanide from the medium, the microbe comprising a lanthanide-dependent alcohol dehydrogenase and an evo-HLn Methylorubrum extorquens AM1 hybrid sensor histidine kinase/response regulator comprising a Leu151His substitution.

2. (canceled)

3. The method of claim 1, wherein the medium comprises a growth substrate selected from methanol, ethanol and glycerol.

4. The method of claim 1, wherein the lanthanide is a heavy lanthanide, selected from gadolinium and europium (atomic numbers 64 and 63, respectively).

5. The method of claim 1, wherein the microbe is a Methylobacteriaceae species, including Methylobacterium species, such as Methylobacterium adhaesivum, Methylobacterium aminovorans, Methylobacterium aquaticum, Methylobacterium chloromethanicum, M. dichloromethanicum, Methylobacterium extorquens, Methylobacterium fujisawaense, Methylobacterium hispanicum, Methylobacterium isbiliense, Methylobacterium lusitanum, Methylobacterium mesophilicum, Methylobacterium nodulans, Methylobacterium organophilum, Methylobacterium podarium, Methylobacterium populi, Methylobacterium radiotolerans, Methylobacterium rhodesianum, Methylobacterium rhodinum, Methylobacterium suomiense, Methylobacterium thiocyanatum, Methylobacterium variabile, Methylobacterium zatmanii; and Methylorubrum species, such as Methylorubrum aminovorans, Methylorubrum extorquens, Methylorubrum podarium, Methylorubrum populi, Methylorubrum pseudosasae, Methylorubrum rhodesianum, Methylorubrum rhodinum, Methylorubrum salsuginis, Methylorubrum suomiense, Methylorubrum thiocyanatum and Methylorubrum zatmanii.

6. The method of claim 1, wherein the microbe is a Methylorubrum extorquens.

7. The method of claim 1, wherein the microbe is an engineered Methylobacteriaceae species (supra), wherein the regulator is transgenic, engineered and/or recombinant.

8. The method of claim 1, wherein the regulator is transgenic to the microbe.

9. The method of claim 1, wherein the regulator is encoded by a regulator gene comprising 452T>A mutation that results in the Leu151His substitution.

10. The method of claim 1, wherein the microbe comprises a genome comprising one or both of SNPs: 69T>C and 114T>C.

11. The method of claim 1, wherein the method further comprises, after acquisition of an amount of the lanthanide, isolating the microbe from the medium.

12. The method of claim 1, wherein the method further comprises isolating the lanthanide from the microbe.

13. The method of claim 1, wherein the microbe is grown under conditions wherein the microbe produces pyrroloquinoline quinone (PQQ).

14. The method of claim 1, wherein the microbe is grown under conditions wherein the microbe produces pyrroloquinoline quinone (PQQ), and the method further comprises isolating the PPQ from the microbe.

15. An engineered microbe for removing a lanthanide from a medium, the microbe comprising a lanthanide-dependent alcohol dehydrogenase and a transgenic, recombinant Methylorubrum extorquens AM1 hybrid sensor histidine kinase/response regulator comprising a Leu151His substitution.

16. The microbe of claim 15, wherein the microbe is a Methylorubrum extorquens.

17. The microbe of claim 15, wherein the microbe is an engineered Methylobacteriaceae species (supra), wherein the regulator is transgenic, engineered and/or recombinant.

18. The microbe of claim 15, wherein the regulator is transgenic to the microbe.

19. The microbe of claim 15, wherein the regulator is encoded by a regulator gene comprising 452T>A mutation that results in the Leu151His substitution.

20. The microbe of claim 15, wherein the microbe comprises a genome comprising one or both of SNPs: 69T>C and 114T>C.

21. Use of a microbe of claim 15 comprising a lanthanide-dependent alcohol dehydrogenase and an evo-HLn Methylorubrum extorquens AM1 hybrid sensor histidine kinase/response regulator comprising a Leu151His substitution, for the acquisition, storage and use of heavy lanthanides.
